(19) United States
(12) Patent Application Publication
(10) Pub. No.: US 2004/0047849 A1
(43) Pub. Date: Mar. 11, 2004

(54) Use of some lactobacillus strains in treating allergy

(75) Inventors: Ching-Hsiang Hsu, Tainan County (TW); Wei-Chih Su, Tainan County (TW)

Correspondence Address: BANNER & WITCOFF, 1001 G STREET N W, SUITE 1100, WASHINGTON, DC, 20001, US

(73) Assignee: GenMont Biotech Inc., Tainan County (TW)

(21) Appl. No.: 10/238,608

(22) Filed: Sep. 11, 2002

(51) Int. Cl. C12N 1/20

(52) U.S. Cl. 424/93.45; 435/252.9

(57) ABSTRACT

The present invention provides a method for treating allergy in a subject comprising administrating said subject with a medicament comprising a lactic acid bacterial strain stimulating INF-γ secretion, which is selected from the group consisting of Lactobacillus plantarum CCRC 12944, Lactobacillus acidophilus CCRC 14079, Lactobacillus rhamnosus CCRC 10940, Lactobacillus paracasei subsp. paracasei CCRC 14023, Lactobacillus delbrueckii subsp. bulgaricus CCRC 12297, Lactobacillus delbrueckii subsp. bulgaricus CCRC 14007, and Lactobacillus delbrueckii subsp. bulgaricus CCRC 14069. A composition for treating allergy comprising the above-mentioned lactic acid bacterial strain is also provided.

### BACKGROUND OF THE INVENTION

1. Field of the Invention

The invention mainly relates to a new use of some Lactobacillus strains in treating allergy.

2. Description of the Related Art

Allergy refers to an acquired potential to develop immunologically mediated adverse reaction to normally innocuous substances. Allergic reaction provokes symptoms such as itching, coughing, wheezing, sneezing, watery eyes, inflammation and fatigue. It is normally believed that allergic reaction includes an early specific immune response and a late inflammatory reaction. It is reported that allergens (e.g. pollens and mite dust) mediate the early phase of allergy by stimulating high affinity immunoglobulin (IgE) receptors. For instance, mast cells and basophils, when stimulated by allergens, will release histamine and cytokines. The cytokines released from mast cells and basophils then mediate the late phase of allergy by recruiting inflammatory cells. It is also reported that the influx of eosinophils, macrophages, lymphocytes, neutrophils and platelets starts the vicious inflammatory cycle. This late phase of allergy amplifies the initial immune response, which in turn triggers the release of more inflammatory cells (Blease et al. Chemokines and their role in airway hyper-reactivity. Respir Res 2000;1:54-61).

Various therapies have been pursued in order to treat the symptoms of allergies. Among them, anti-allergics and histamine H-receptor antagonists (anti-histamines) have been used. Histamine antagonists are administered to antagonize the action of histamine released from mast cells in response to the presence of allergens. They reduce the redness, itching and swelling caused by the action of histamine on the target tissues, and serve to prevent or alleviate many of the symptoms resulting from degranulation of mast cells. However, anti-histamines have also been associated with adverse reactions such as diminished alertness, slowed reaction times and somnolence (U.S. Pat. No. 6,225,332).

There are also some reports on the treatment of allergies by regulating cytokines. Among them, interferon-γ (INF-γ) was found to inhibit the over-expression of cytokines in Th2 lymphocytes, especially the secretion of IL-4 to lower the proliferation of B cells. Besides, INF-γ could stimulate the immune response of Th1 and repress the synthesis of IgE (Sareneva T et al. Influenza A virus-induced INF-α/β and IL-18 synergistically enhance IFN-γ gene expression in human T cells. J Immunol 1998; 160:6032-6038; Shida K et al. Lactobacillus casei inhibits antigen-induced IgE secretion through regulation of cytokine production in murine splenocyte culture. Int Arch Allergy Immunol 1998;115:278-287). Since INF-γ can repress B cell proliferation and IgE secretion, it is believed that INF-γ is effective in treating allergy.

Lactic acid bacteria, which are gram-positive bacteria, are commonly used in industrial food fermentations. In recent studies, lactic acid bacteria were shown to stimulate INF-γ secretion of cells (Contractor NV et al. Lymphoid hyperplasia, autoimmunity and compromised intestinal intraepithelial lymphocyte development in colits-free gnotobiotic IL-2-deficient mice. J Immunol 1998; 160:385-394). Some specific lactic acid bacteria, such as Bifidobacterium lactis and Lactobacillus brevis subsp., were found to stimulate INF-γ secretion of lymphocytes in blood derived from mice and humans (U.S. patent Publication Ser. No. 2002/0,031,503 A1; U.S. Pat. No. 5,556,785). It was also reported that lactic acid bacteria could stimulate lymphocytes derived from humans or mice to secret Interleukin-12 (IL-12), which was a T cell stimulatory cytokine activating T cells and NK cells to secrete INF-γ (Hessle et al. Lactobacilli from human gastrointestinal mucosa are strong stimulators of IL-12 production. Clin Exp Immunol 1999; 116:276-282).

### SUMMARY OF THE INVENTION

The invention provides a new use of some Lactobacillus strains in treating allergy.

One subject of the invention is to provide a method for treating allergy in a subject comprising administrating said subject with a medicament comprising a lactic acid bacterial strain stimulating INF-γ secretion, which is selected from the group consisting of Lactobacillus plantarum CCRC 12944, Lactobacillus acidophilus CCRC 14079, Lactobacillus rhamnosus CCRC 10940, Lactobacillus paracasei subsp. paracasei CCRC 14023, Lactobacillus delbrueckii subsp. bulgaricus CCRC 12297, Lactobacillus delbrueckii subsp. bulgaricus CCRC 14007, and Lactobacillus delbrueckii subsp. bulgaricus CCRC 14069.

In another aspect, the invention provides a composition for treating allergy comprising a lactic acid bacterial strain stimulating INF-γ secretion in a therapeutically effective amount to treat allergy, which strain is selected from the group consisting of Lactobacillus plantarum CCRC 12944, Lactobacillus acidophilus CCRC 14079, Lactobacillus rhamnosus CCRC 10940, Lactobacillus paracasei subsp. paracasei CCRC 14023, Lactobacillus delbrueckii subsp. bulgaricus CCRC 12297, Lactobacillus delbrueckii subsp. bulgaricus CCRC 14007, and Lactobacillus delbrueckii subsp. bulgaricus CCRC 14069.

### BRIEF DESCRIPTION OF THE DRAWINGS

FIG. 1 illustrates the secretion of INF-γ in the co-culture of a Lactobacillus strain and lymphocytes. The secretions of INF-γ were detected with ELISA after the 12 and 36-hour co-culture of the lactic acid bacterium and lymphocytes, respectively. The amounts of INF-γ were expressed by the absorbance values (O.D. values). In the figure, “PC” represents Lactobacillus casei CCRC 10697 as positive control; “NC” represents Lactobacillus delbrueckii subsp. bulgaricus CCRC 14071 as negative control; 1 represents Lactobacillus plantarum CCRC 12944; 2 represents Lactobacillus acidophilus CCRC 14079; 3 represents Lactobacillus rhamnosus CCRC 10940; 4 represents Lactobacillus paracasei subsp. paracasei CCRC 14023; 5 represents Lactobacillus delbrueckii subsp. bulgaricus CCRC 12297; 6 represents Lactobacillus delbrueckii subsp. bulgaricus CCRC 14007; and 7 represents Lactobacillus delbrueckii subsp. bulgaricus CCRC 14069.

FIG. 2 illustrates the secretion of INF-γ in the co-culture of a Lactobacillus strain and peripheral blood mononuclear cells (PBMC). The secretions of INF-γ were detected with ELISA after the 12, 48, and 72-hour co-culture of the lactic acid bacterium and PBMCs, respectively. The amounts of INF-γ were expressed by the absorbance values (O.D. values). In the test, Lactobacillus casei CCRC 10697 was used as positive control; Lactobacillus delbrueckii subsp. bulgaricus CCRC 14071 was used as negative control; Lactobacillus paracasei subsp. paracasei CCRC 14023 was tested.

### DETAILED DESCRIPTION OF THE INVENTION

According to the invention, some Lactobacillus strains stimulating INF-γ secretion are unexpectedly found, and can be used for treating allergy.

In one aspect, the invention provides a method for treating allergy in a subject comprising administrating said subject with a medicament comprising a lactic acid bacterial strain stimulating INF-γ secretion, which is selected from the group consisting of Lactobacillus plantarum CCRC 12944, Lactobacillus acidophilus CCRC 14079, Lactobacillus rhamnosus CCRC 10940, Lactobacillus paracasei subsp. paracasei CCRC 14023, Lactobacillus delbrueckii subsp. bulgaricus CCRC 12297, Lactobacillus delbrueckii subsp. bulgaricus CCRC 14007, and Lactobacillus delbrueckii subsp. bulgaricus CCRC 14069, which are all deposited at the Food Industry Research and Development Institute (FIRDI), Hsinchu, Taiwan. The above-mentioned strains became available to the public from the FIRDI. They are safe, natural, nontoxic, and meet the G.R.A.S. (General Regarded as Safe) standard. The strains are commonly used in food and are not harmful to humans.

According to the invention, the strains were proved to have the ability to stimulate INF-γ secretion when co-incubated with lymphocytes. One of the strains was proved to stimulate INF-γ secretion of the peripheral blood mononuclear cells (PBMCs). In the most preferred embodiment of the invention, Lactobacillus paracasei subsp. paracasei CCRC 14023 was found to have a better (four-fold) ability to stimulate INF-γ secretion than Lactobacillus casei CCRC 10697 as positive control.

According to the invention, the lactic acid bacterial strain used in the treatment of allergy can be live or inactive. For instance, the live bacterial strains can be treated with a heating step or other treatments commonly used in the art for killing the lactic acid bacterial strains to obtain inactive strains.

The term “allergy” used herein refers to INF-γ mediated allergy. The allergic disorders include rhinitis, sinusitis, asthma, hypersensitive pneumonia, extrinsic allergic alveolitis, conjunctivitis, urticaria, eczema, dermatitis, anaphylaxis, angioedema, allergic and migraine headache, and certain gastrointestinal disorders. It has been proven that atopic eczema is treatable with the probiotics stimulating INF-γ secretion (Isolauri E et al. Probiotics in the management of atopic eczema. Clinical and experimental Allergy 2000; 30:1604-1610; Sutas Y et al. Suppression of lymphocyte proliferation in vitro by bovine caseins hydrolyzed with Lactobacillus casei GG-derived enzyme. J Allergy Clin Immunol 1996; 98:216-224; Kalliomaki M et al. Probiotics in primary prevention of atopic disease: a randomized placebo-controlled trial. Lancet 2001; 357:1076-79).

In another aspect, the invention provides a composition for treating allergy comprising a lactic acid bacterial strain stimulating INF-γ secretion in a therapeutically effective amount to treat allergy, which strain is selected from the group consisting of Lactobacillus plantarum CCRC 12944, Lactobacillus acidophilus CCRC 14079, Lactobacillus rhamnosus CCRC 10940, Lactobacillus paracasei subsp. paracasei CCRC 14023, Lactobacillus delbrueckii subsp. bulgaricus CCRC 12297, Lactobacillus delbrueckii subsp. bulgaricus CCRC 14007, and Lactobacillus delbrueckii subsp. bulgaricus CCRC 14069.

According to the invention, the lactic acid bacterial strain can be included in a pharmaceutical composition, dietary supplement, food or the components thereof, which are normally administrated by people. In a preferred embodiment of the invention, the lactic acid bacterial strain can be delivered in food form, such as in a coagulated milk product that prepared through the fermentation of lactic acid in milk. The food products prepared according to the invention can be conveniently administrated to infants or children.

The following Examples are given for the purpose of illustration only and are not intended to limit the scope of the present invention.

### EXAMPLE 1

Screening of Lactic Acid Bacterial Strains Stimulating INF-γ Secretion in Lymphocytes

Bacterial Culture: Sixty-seven lactic acid bacterial strains listed in Table 1 were pre-selected. The strains as positive control (PC) and negative control (NC) were also illustrated. All strains were purchased from the FIRDI.

TABLE 1

| No. | Lactic acid bacterial strain | CCRC No. |
|---|---|---|
| PC | Lactobacillus casei | 10697 |
| NC | Lactobacillus delbrueckii subsp. bulgaricus | 14071 |
| 1 | Lactobacillus plantarum | 10069 |
| 2 | Lactobacillus plantarum | 10357 |
| 3 | Lactobacillus plantarum | 11697 |
| 4 | Lactobacillus plantarum | 12250 |
| 5 | Lactobacillus plantarum | 12251 |
| 6 | Lactobacillus plantarum | 12327 |
| 7 | Lactobacillus plantarum | 12944 |
| 8 | Lactobacillus plantarum | 14059 |
| 9 | Lactobacillus plantarum | 15478 |
| 10 | Lactobacillus johnsonii | 14004 |
| 11 | Lactobacillus acidophilus | 14026 |
| 12 | Lactobacillus rhamnosus | 14029 |
| 13 | Lactobacillus acidophilus | 14064 |
| 14 | Lactobacillus acidophilus | 14065 |
| 15 | Lactobacillus acidophilus | 14079 |
| 16 | Lactobacillus sp. | 16000 |
| 17 | Lactobacillus acidophilus | 16092 |
| 18 | Lactobacillus acidophilus | 16099 |
| 19 | Lactobacillus acidophilus | 17009 |
| 20 | Lactobacillus acidophilus | 17064 |
| 21 | Lactobacillus acidophilus | 10695 |
| 22 | Lactobacillus casei subsp. casei | 10358 |
| 23 | Lactobacillus rhamnosus | 10940 |
| 24 | Lactobacillus casei subsp. casei | 11197 |
| 25 | Lactobacillus rhamnosus | 11673 |
| 26 | Lactobacillus paracasei subsp. paracasei | 12193 |
| 27 | Lactobacillus paracasei subsp. paracasei | 12248 |
| 28 | Lactobacillus casei subsp. casei | 12249 |
| 29 | Lactobacillus casei subsp. casei | 12272 |
| 30 | Lactobacillus paracasei subsp. paracasei | 14001 |
| 31 | Lactobacillus paracasei subsp. paracasei | 14023 |
| 32 | Lactobacillus casei subsp. casei | 14025 |
| 33 | Lactobacillus casei subsp. casei | 14073 |
| 34 | Lactobacillus casei subsp. casei | 14074 |
| 35 | Lactobacillus casei subsp. casei | 14080 |
| 36 | Lactobacillus casei subsp. casei | 14082 |
| 37 | Lactobacillus casei subsp. casei | 14083 |
| 38 | Lactobacillus casei subsp. casei | 14084 |
| 39 | Lactobacillus casei subsp. casei | 14705 |
| 40 | Lactobacillus casei subsp. casei | 16093 |
| 41 | Lactobacillus casei subsp. casei | 16094 |
| 42 | Lactobacillus paracasei subsp. paracasei | 16100 |
| 43 | Lactobacillus casei subsp. casei | 17001 |
| 44 | Lactobacillus casei subsp. casei | 17002 |
| 45 | Lactobacillus casei subsp. casei | 17004 |
| 46 | Lactobacillus casei subsp. casei | 17005 |
| 47 | Lactobacillus delbrueckii subsp. bulgaricus | 10696 |
| 48 | Lactobacillus helveticus | 11052 |
| 49 | Lactobacillus delbrueckii subsp. bulgaricus | 12255 |
| 50 | Lactobacillus delbrueckii subsp. bulgaricus | 12297 |
| 51 | Lactobacillus delbrueckii subsp. bulgaricus | 14007 |
| 52 | Lactobacillus delbrueckii subsp. bulgaricus | 14008 |
| 53 | Lactobacillus delbrueckii subsp. bulgaricus | 14009 |
| 54 | Lactobacillus delbrueckii subsp. bulgaricus | 14010 |
| 55 | Lactobacillus delbrueckii subsp. bulgaricus | 14069 |
| 56 | Lactobacillus delbrueckii subsp. bulgaricus | 14075 |
| 57 | Lactobacillus delbrueckii subsp. bulgaricus | 14077 |
| 58 | Lactobacillus delbrueckii subsp. bulgaricus | 14090 |
| 59 | Lactobacillus delbrueckii subsp. bulgaricus | 14091 |
| 60 | Lactobacillus delbrueckii subsp. bulgaricus | 14098 |
| 61 | Lactobacillus deibrueckii subsp. bulgaricus | 16050 |
| 62 | Lactobacillus delbrueckii subsp. bulgaricus | 16051 |
| 63 | Lactobacillus delbrueckii subsp. bulgaricus | 16052 |
| 64 | Lactobacillus delbrueckii subsp. bulgaricus | 16053 |
| 65 | Lactobacillus paracasei subsp. paracasei | 12188 |
| 66 | Lactobacillus brevis | 12247 |
| 67 | Lactobacillus brevis | 14060 |

Among them, thirty-eight strains were safe, natural, nontoxic, and met the G.R.A.S. (General Regarded as Safe) standard. All of the strains were cultured in Lactobacillus MRS broth (DIFCO 0881) at 37° C. to the stationary phase, and collected by centrifuging at 3000 g for 15 minutes and washed with 2 mL and 1 mL PBS (phosphate buffered saline, pH 7.2). The cultures of the strains were re-suspended in 1 mL PBS and then heated at 95° C. for 30 minutes, and then were autoclaved and stored in PBS at −20° C.

Lymphocyte Culture: HL-60 CCRC 60273 (Clone 15 HL-60) cells (purchased from the FIRDI) were treated according to the method described by Fischkoff (Fischkoff S. A. Graded increase in probability of eosinophilic differentiation of HL-60 promyelocytic leukemia cells induced by culture under alkaline condition. Leukemia Research 1988; 12(8): 679-686). The HL-60 cells were subcultured in RPMI 1640 (pH 7.2) and induced to differentiate to eosinophils and then subcultured with RPMI 1640 (pH 7.7) for several generations to obtain lymphocyte samples. In each lymphocyte sample, the cell density was adjusted to 5×10⁶ cells per sample. The lymphocyte samples were incubated in 2 mL RPMI 1640 (pH 7.7) for 6 hours.

Stimulating INF-γ Secretion: The lymphocyte samples were co-cultured with a given amount of the above-mentioned bacterial strains. Lactobacillus casei CCRC 10697 was taken as a positive control and Lactobacillus delbrueckii subsp. bulgaricus CCRC 14071 as a negative control. After the 12, 36 and 60-hour co-culture, the cells in each sample were collected, respectively. The collected cells were re-suspended and centrifuged at 2000 rpm for 5 minutes. The supernatant were taken for the determination of the INF-γ level in each sample.

Determination of INF-γ Level: The method for determining INF-γ level by ELISA was described by Shida et al. (Shida K., Makino K., Morishita A., Takamizawa K., Hachimura S., Ametani A., Takehito S., Kumagai Y., Habu S., Kaminogawa S. Lactobacillus casei inhibits antigen induced IgE secretion through regulation of cytokine production in murine splenocyte cultures. Int Arch Allergy Immunol 1998; 115:278-287) comprising the steps of:

adding 150 μL of 2.5 μg/mL purified mouse anti-human INF-γ antibodies in coating buffer (8.00 g NaCl, 0.20 g KCl, 1.44 g Na₂HPO₄, 0.24 g KH₂PO₄, 30.0 g bovine serum albumin, and 0.50 g NaN₃ per liter, pH 7.4) into each well of an ELISA plate;

shaking the plate at 40 rpm at room temperature;

incubating the plate at 4° C. overnight;

discarding the coating buffer in the wells;

washing each well of the plate with wash buffer (8.00 g NaCl, 0.20 g KCl, 1.44 g Na₂HPO₄, 0.24 g KH₂PO₄, 0.5 mL Tween 20, and 0.50g NaN₃ per liter, pH 7.4) for 3 minutes twice;

washing the wells with distilled water;

adding 200 μL block buffer into each well of the plate;

incubating the plate at room temperature for at least 2 hours;

discarding the block buffer in the wells;

washing each well of the plate with wash buffer for 3 minutes three times;

washing each well of the plate with distilled water;

taking the supernatant of the lymphocyte sample and adding it to each well of the plate;

shaking the plate at 40 rpm at 4° C. overnight;

discarding the samples in the wells;

washing each well of the plate with wash buffer for 3 minutes three times and then with distilled water;

adding 150 μL biotin mouse anti-human INF-γ antibodies diluted with dilute buffer into each well of the plate;

incubating the plate for 2 hours at room temperature;

washing each well of the plate with wash buffer for 3 minutes three times and then with distilled water;

adding 150 μL Streptavidin-Alkaline phosphatase (Streptavidin-AKP) diluted with dilute buffer into each well of the plate;

incubating the plate for 1 hour at room temperature;

washing each well of the plate with wash buffer for 3 minutes four times and then with distilled water;

adding 150 μL of substrate p-Nitrophenyl phosphate (pNpp) into each well of the plate;

incubating the plates at room temperature until the substrate reaction is completed;

measuring the absorbance of each well of the plate at 405 nm (i.e. OD405).

Result: The results of INF-γ level stimulated by the 68 lactic acid bacterial strains are listed in Table 2.

TABLE 2

| CCRC No. | 12 hours (OD) | 36 hours (OD) | 60 hours (OD) |
|---|---|---|---|
| Positive Control | 0.156 | 0.295 | 0.106 |
| Negative Control | 0.117 | 0.241 | 0.103 |
| 10069 | 0.117 | 0.304 | 0.107 |
| 10357 | 0.129 | 0.267 | 0.104 |
| 11697 | 0.112 | 0.397 | 0.104 |
| 12250 | 0.122 | 0.335 | 0.156 |
| 12251 | 0.177 | 0.293 | 0.110 |
| 12327 | 0.131 | 0.289 | 0.111 |
| 12944 | 0.152 | 0.427 | 0.092 |
| 14059 | 0.111 | 0.363 | 0.102 |
| 15478 | 0.157 | 0.385 | 0.109 |
| 14004 | 0.162 | 0.399 | 0.106 |
| 14026 | 0.115 | 0.405 | 0.103 |
| 14029 | 0.131 | 0.272 | 0.110 |
| 14064 | 0.114 | 0.337 | 0.164 |
| 14065 | 0.159 | 0.244 | 0.110 |
| 14079 | 0.142 | 0.342 | 0.099 |
| 16000 | 0.123 | 0.255 | 0.105 |
| 16092 | 0.127 | 0.254 | 0.114 |
| 16099 | 0.114 | 0.262 | 0.114 |
| 17009 | 0.111 | 0.276 | 0.117 |
| 17064 | 0.147 | 0.272 | 0.114 |
| 10695 | 0.131 | 0.274 | 0.118 |
| 10358 | 0.148 | 0.271 | 0.119 |
| 10697 | 0.160 | 0.340 | 0.098 |
| 10940 | 0.336 | 0.335 | 0.109 |
| 11197 | 0.150 | 0.293 | 0.104 |
| 11673 | 0.109 | 0.298 | 0.106 |
| 12193 | 0.116 | 0.305 | 0.111 |
| 12248 | 0.160 | 0.284 | 0.112 |
| 12249 | 0.142 | 0.267 | 0.112 |
| 12272 | 0.120 | 0.276 | 0.112 |
| 14001 | 0.173 | 0.410 | 0.108 |
| 14023 | 0.120 | 0.538 | 0.125 |
| 14025 | 0.142 | 0.339 | 0.110 |
| 14073 | 0.157 | 0.398 | 0.104 |
| 14074 | 0.125 | 0.455 | 0.117 |
| 14080 | 0.124 | 0.308 | 0.116 |
| 14082 | 0.148 | 0.248 | 0.113 |
| 14083 | 0.129 | 0.203 | 0.116 |
| 14084 | 0.153 | 0.335 | 0.121 |
| 14705 | 0.159 | 0.277 | 0.122 |
| 16093 | 0.131 | 0.328 | 0.127 |
| 16094 | 0.160 | 0.309 | 0.114 |
| 16100 | 0.158 | 0.316 | 0.121 |
| 17001 | 0.219 | 0.252 | 0.123 |
| 17002 | 0.155 | 0.207 | 0.120 |
| 17004 | 0.236 | 0.112 | 0.119 |
| 17005 | 0.125 | 0.320 | 0.104 |
| 10696 | 0.122 | 0.373 | 0.122 |
| 11052 | 0.142 | 0.316 | 0.107 |
| 12255 | 0.118 | 0.325 | 0.116 |
| 12297 | 0.121 | 0.418 | 0.105 |
| 14007 | 0.122 | 0.502 | 0.110 |
| 14008 | — | 0.359 | 0.100 |
| 14009 | 0.224 | 0.293 | 0.103 |
| 14010 | 0.150 | 0.312 | 0.100 |
| 14069 | 0.146 | 0.440 | 0.161 |
| 14071 | 0.144 | 0.270 | 0.099 |
| 14075 | 0.152 | 0.319 | 0.100 |
| 14077 | 0.163 | 0.342 | 0.102 |
| 14090 | 0.203 | 0.302 | 0.106 |
| 14091 | 0.184 | 0.288 | 0.097 |
| 14098 | 0.147 | 0.242 | 0.101 |
| 16050 | 0.136 | 0.264 | 0.098 |
| 16051 | 0.135 | 0.250 | 0.103 |
| 16052 | 0.132 | 0.386 | 0.104 |
| 16053 | 0.132 | 0.314 | 0.113 |
| 12188 | 0.150 | 0.263 | 0.101 |
| 12247 | 0.137 | 0.246 | 0.103 |
| 14060 | 0.167 | 0.328 | 0.103 |

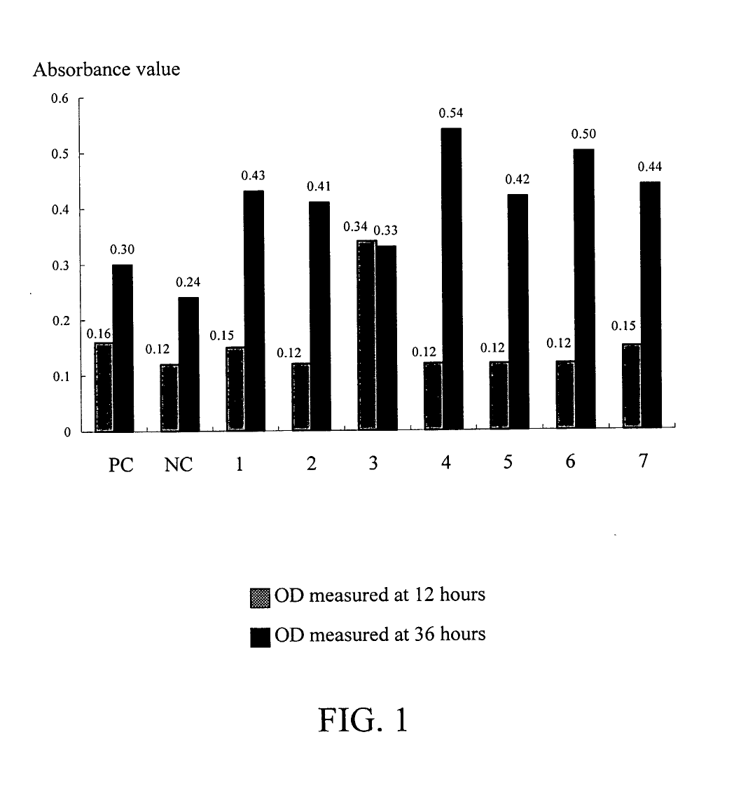

Among the 67 strains, the following seven strains were found to be capable of stimulating INF-γ secretion in lymphocyte cells: Lactobacillus plantarum CCRC 12944, Lactobacillus acidophilus CCRC 14079, Lactobacillus rhamnosus CCRC 10940, Lactobacillus paracasei subsp. paracasei CCRC 14023, and Lactobacillus delbrueckii subsp. bulgaricus CCRC 12297, 14007 and 14069. The results are shown in FIG. 1. The OD405 values of Lactobacillus paracasei subsp. paracasei CCRC 14023, and Lactobacillus delbrueckii subsp. bulgaricus CCRC 12297, 14007 and 14069 are higher than the positive control, and even higher than the negative control by four folds. Besides, the OD405 values of the strains except Lactobacillus rhamnosus (CCRC 10940) collected after the 36-hour co-culture are 3-fold higher than those after 12-hour co-culture.

### EXAMPLE 2

INF-γ Secretions in Peripheral Blood Mononuclear Cells by Stimulation of Lactic Acid Bacteria

Isolation of Peripheral Blood Mononuclear Cells: Five mL blood samples derived from healthy volunteers were added with 5 mL Ficoll-Hypaque (17-1400-02, Pharmacia) and then centrifuged at 500 g for 30 minutes. The peripheral blood mononuclear cells (PBMCs) were taken from the interface of the samples, and washed twice with PBS. The PBMCs (10⁵ cells/mL) were transferred to the wells of a six-well plate wherein each well contained 2 mL RPMI 1640 medium of pH 7.7.

Stimulating INF-γ Secretion: Using the analogous method described in Example 1, the PBMCs were co-cultured with Lactobacillus plantarum CCRC 12944, Lactobacillus acidophilus CCRC 14079, Lactobacillus rhamnosus CCRC 10940, Lactobacillus paracasei subsp. paracasei CCRC 14023, and Lactobacillus delbrueckii subsp. bulgaricus CCRC 12297, 14007 and 14069 (10⁷ cells/mL). Lactobacillus casei CCRC 10697 was taken as a positive control and Lactobacillus delbrueckii subsp. bulgaricus CCRC 14071 was taken as a negative control. The cells were collected after the 24, 48 and 72-hour co-culture, and re-suspended and centrifuged at 2000 rpm for 5 minutes. The supernatant were taken for determining the INF-γ levels by the same method described in Example 1.

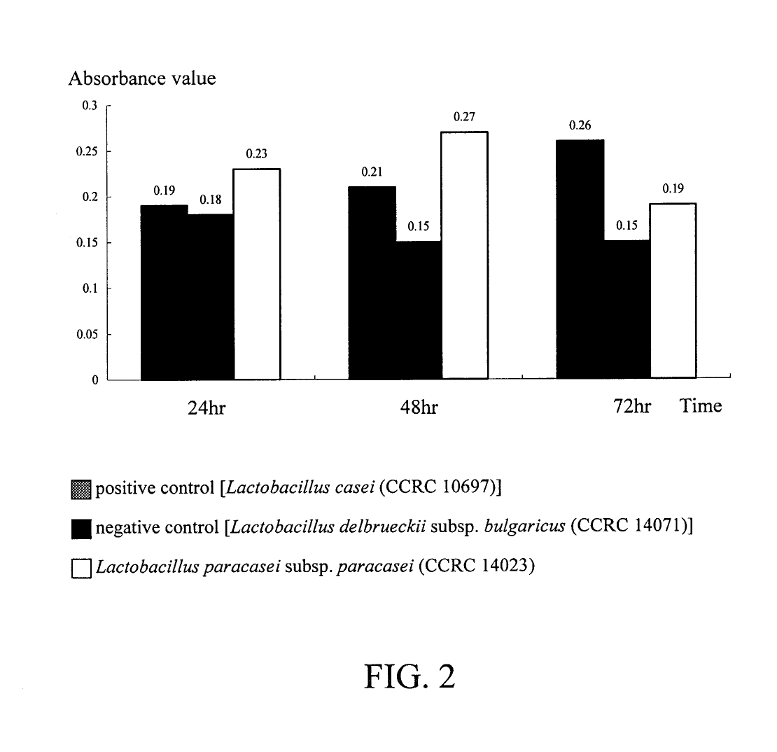

Results: The Results of the amount of INF-γ of PBMCs stimulated by the seven strains are listed in Table 3, and especially, the result by Lactobacillus paracasei subsp. paracasei CCRC 14023 is shown in FIG. 2.

TABLE 3

| | | | INF-γ conc. |
|---|---|---|---|
| Time | CCRC Nos. | OD | (ng/ml) |
| 24 hours | Positive control | 0.1945 | 861.5 |
| | 12944 | 0.1685 | 731.5 |
| | 14079 | 0.1895 | 836.5 |
| | 10940 | 0.223 | 1004 |
| | 14023 | 0.23 | 1039 |
| | 12297 | 0.195 | 864 |
| | 14007 | 0.165 | 714 |
| | 14069 | 0.2015 | 896.5 |
| 48 hours | Positive control | 0.2095 | 936.5 |
| | 12944 | 0.1605 | 691.5 |
| | 14079 | 0.244 | 1109 |
| | 10940 | 0.305 | 1414 |
| | 14023 | 0.267 | 1224 |
| | 12297 | 0.1555 | 666.5 |
| | 14007 | 0.141 | 594 |
| | 14069 | 0.165 | 714 |
| 72 hours | Positive control | 0.2575 | 1176.5 |
| | 12944 | 0.159 | 684 |
| | 14079 | 0.17 | 739 |
| | 10940 | 0.193 | 854 |
| | 14023 | 0.1895 | 836.5 |
| | 12297 | 0.147 | 624 |
| | 14007 | 0.133 | 554 |
| | 14069 | 0.17 | 739 |

The OD405 value of the sample collected after the 24-hour co-culture is 1.2-fold higher than the negative control; that collected after the 48-hour co-culture is 1.8-fold higher than the negative control and 1.3-fold higher than the positive control; and that collected after the 72-hour co-culture is 1.3-fold higher than the negative control.

While embodiments of the present invention have been illustrated and described, various modifications and improvements can be made by persons skilled in the art. It is intended that the present invention is not limited to the particular forms as illustrated, and that all the modifications not departing from the spirit and scope of the present invention are within the scope as defined in the appended claims.

What is claimed is:

1. A method for treating allergy in a subject comprising administrating said subject with a medicament comprising a lactic acid bacterial strain stimulating INF-γ secretion, which is selected from the group consisting of Lactobacillus plantarum CCRC 12944, Lactobacillus acidophilus CCRC 14079, Lactobacillus rhamnosus CCRC 10940, Lactobacillus paracasei subsp. paracasei CCRC 14023, Lactobacillus delbrueckii subsp. bulgaricus CCRC 12297, Lactobacillus delbrueckii subsp. bulgaricus CCRC 14007, and Lactobacillus delbrueckii subsp. bulgaricus CCRC 14069, which are all deposited at the Food Industry Research and Development Institute (FIRDI), Hsinchu, Taiwan.

2. The method of claim 1, wherein the lactic acid bacterial strain is Lactobacillus plantarum CCRC 12944.

3. The method of claim 1, wherein the lactic acid bacterial strain is Lactobacillus acidophilus CCRC 14079.

4. The method of claim 1, wherein the lactic acid bacterial strain is Lactobacillus rhamnosus CCRC 10940.

5. The method of claim 1, wherein the lactic acid bacterial strain is Lactobacillus paracasei subsp. paracasei CCRC 14023.

6. The method of claim 1, wherein the lactic acid bacterial strain is Lactobacillus delbrueckii subsp. bulgaricus CCRC 12297.

7. The method of claim 1, wherein the lactic acid bacterial strain is Lactobacillus delbrueckii subsp. bulgaricus CCRC 14007.

8. The method of claim 1, wherein the lactic acid bacterial strain is Lactobacillus delbrueckii subsp. bulgaricus CCRC 14069.

9. The method of claim 1, wherein the lactic acid bacterial strain is live or inactivated.

10. The method of claim 9, wherein the lactic acid bacterial strain is inactivated.

11. A composition for treating allergy comprising a lactic acid bacterial strain stimulating INF-γ secretion in a therapeutically effective amount to stimulate INF-γ secretion, which strain is selected from the group consisting of Lactobacillus plantarum CCRC 12944, Lactobacillus acidophilus strain CCRC 14079, Lactobacillus rhamnosus strain CCRC 10940, Lactobacillus paracasei subsp. paracasei strain CCRC 14023, Lactobacillus delbrueckii subsp. bulgaricus strain CCRC 12297, Lactobacillus delbrueckii subsp. bulgaricus strain CCRC 14007, and Lactobacillus delbrueckii subsp. bulgaricus strain CCRC 14069, which are all deposited at the Food Industry Research and Development Institute (FIRDI), Hsinchu, Taiwan.

12. The composition of claim 11, wherein the lactic acid bacterial strain is Lactobacillus plantarum CCRC 12944.

13. The composition of claim 11, wherein the lactic acid bacterial strain is Lactobacillus acidophilus CCRC 14079.

14. The composition of claim 11, wherein the lactic acid bacterial strain is Lactobacillus rhamnosus CCRC 10940.

15. The composition of claim 11, wherein the lactic acid bacterial strain is Lactobacillus paracasei subsp. paracasei CCRC 14023.

16. The composition of claim 11, wherein the lactic acid bacterial strain is Lactobacillus delbrueckii subsp. bulgaricus CCRC 12297.

17. The composition of claim 11, wherein the lactic acid bacterial strain is Lactobacillus delbrueckii subsp. bulgaricus CCRC 14007.

18. The composition of claim 11, wherein the lactic acid bacterial strain is Lactobacillus delbrueckii subsp. bulgaricus CCRC 14069.

19. The composition of claim 11, wherein the lactic acid bacterial strain is live or inactivated.

20. The composition of claim 19, wherein the lactic acid bacterial strain is inactivated.

21. The composition of claim 11 in the form of a pharmaceutical composition, dietary supplement, food, or the component thereof.
